(19) United States
(12) Patent Application Publication
(10) Pub. No.: US 2008/0195131 A1
(43) Pub. Date: Aug. 14, 2008

(54) Lancing Device End Cap With Rocking-Actuated Surface Features

(75) Inventors: Stephen J. Flynn, Peachtree City, GA (US); Richard W. LeVaughn, Newman, GA (US); Christopher J. Ruf, Marietta, GA (US); Gwenn E. Kennedy, Ellenwood, GA (US); Michael V. Lipoma, Villa Rica, GA (US); Juan D. Salleras, Alpharetta, GA (US); Jac Holzaepfel, Peachtree City, GA (US); Sylvia Ramos, Marietta, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, Marietta, GA (US)

(21) Appl. No.: 10/572,267

(22) PCT Filed: Jul. 28, 2004
(86) PCT No.: PCT/US04/24407

§ 371 Date: Jul. 24, 2007

(51) Int. Cl. A61B 5/151

(52) U.S. Cl. 606/181

(57) ABSTRACT

An endcap for a lancing device, having a contact face defining an opening, wherein the contact face includes one or more surface contour features for directing body fluid toward the opening upon rocking of the lancing device with the contact face in contact with the skin surrounding the lancing site.

### CROSS-REFERENCE TO RELATED APPLICATION

This application claims the benefit of U.S. Provisional Patent Application Ser. No. 60/503,786, filed Sep. 18, 2003, the entirety of which is hereby incorporated herein by reference for all purposes.

### TECHNICAL FIELD

The present invention relates generally to medical devices and procedures, and more particularly to lancing devices for sampling blood or other bodily fluids. The invention more particularly relates to an end cap for use in conjunction with a lancing device that allows for the collection of an increased volume of blood or bodily fluid to be sampled.

### BACKGROUND OF THE INVENTION

Many medical procedures require puncturing of the skin, and sometimes underlying tissues, of a human or animal subject. For example, a sharp lancet tip is commonly used to puncture the subject's skin for sampling of blood or other bodyfluid, as for example in blood glucose monitoring by diabetics. Generally, lancet blades or needles are much thinner than traditional hypodermic syringe needles, and therefore result in less pain to the patient. A lancet having a sharp lancing tip typically is mounted, permanently or releasably, to the drive mechanism of a lancing device. The drive mechanism includes one or more springs, cams, motors, or other mechanism(s) for moving the lancing tip from a retracted position shielded within a housing of the lancing device to an extended position outside of the housing for lancing the subjects skin. The lancing device may also include a cocking mechanism for arming the drive mechanism, and/or a triggering mechanism for firing the device. The housing of the lancing device typically includes an endcap with an opening through which the lancing tip extends in its extended position. The endcap may be releasably or permanently attached to the remainder of the housing, or can comprise an integral part of the housing.

One common problem that has been found to exist with many known lancets and lancing devices is the closure of the puncture site before an adequate volume of blood has been collected. Premature closure of the puncture requires additional blood to be drawn from the patient, often in a separate and sometimes less desirable location, resulting in an increase in pain and stress to the subject. Costs also increase, as multiple lancets are required to acquire an adequate sample. This is particularly the case when alternate lancing sites other than the fingertips, such as for example the forearm or earlobe, are lanced. Lancing at such alternate lancing sites may be desirable because repeated lancing of the fingertips can result in callous formation, leading to less consistent sampling and decreased sensitivity of the fingertips. In addition, the fingertips have a greater density of nerve endings than alternate lancing sites, resulting in greater pain sensation from fingertip lancing than lancing at alternate sites. To increase patient compliance with a prescribed sampling regimen, it is desirable to minimize pain resulting from the lancing operation. However, because alternate sites typically contain a lower density of capillaries or a deeper capillary bed than the fingertips, alternate lancing sites often yield insufficient sample sizes or are prone to premature closure.

Attempts have been made to provide lancing devices that inhibit premature closure of the puncture site and yield increased sample size. Such attempts have included the use of a stimulator member slideably mounted to the housing for cyclically pumping around the puncture site to “milk” the wound. Although utilizing the aforementioned pumping sequence may allow a larger sample to be collected before closure of the puncture site, occasionally a sample is still insufficient, and another lancing operation to collect blood or analytic fluid is required. Additionally, the repeated application and release of pressure from such pumping can result in contact between the sample and the lancing device, smearing and/or contaminating the sample and necessitating another lancing operation, and/or can lead to bruising of the tissue surrounding the lancing site. Also, the provision of a translationally mounted stimulator member results in increased complexity and cost of the lancing device.

Thus it can be seen that needs exist for improved lancing devices and methods to facilitate increased sample volume of blood or other analytic fluid, and to prevent premature closure of the sampling site. Needs further exist for such mechanisms and methods that are readily adaptable to current lancing devices and procedures, and which can be utilized for lancing at fingertip as well as alternate sample collection sites.

### SUMMARY OF THE INVENTION

In example forms, the present invention is an endcap for a lancing device that enables collection of an increased volume of blood or other body fluid to be sampled from a lancing site of a human or animal subject. The device of the present invention is suitable for use at fingertip lancing sites, but is also well suited for use at alternate lancing sites such as the forearm and/or earlobe. In further embodiments, the invention is a sampling method for increasing collected sample size of blood or other body fluid from a lancing site.

In one aspect, the present invention is an endcap for a lancing device. The endcap preferably includes an opening for passage of at least a sharp tip portion of a lancet, a contact face surrounding the opening, and at least one surface feature on the contact face adjacent the opening for applying pressure to tissue surrounding a lancing site by rocking of the contact face in contact with the tissue surrounding the lancing site.

In another aspect, the invention is an endcap for a lancing device, the endcap preferably including an outwardly-curved contact face for rocking contact with tissue surrounding a lancing site, and at least one slotted opening through the outwardly-curved contact face, the slotted opening being narrower at a central portion than at extremities thereof.

In another aspect, the invention is a lancing device for collecting a sample of body fluid from a sampling site on the skin of a subject. The lancing device preferably includes a lancet and a housing comprising an endcap, the lancet being movable between a first position within the housing and a second position wherein at least a sharp tip portion of the lancet extends through an opening in the endcap. The endcap preferably includes a contact face surrounding the opening and at least one surface feature on the contact face adjacent the opening for applying pressure to tissue surrounding the sampling site by rocking of the contact face in contact therewith.

In still another aspect, the invention is a lancing device for collecting a sample of body fluid from a sampling site on the skin of a subject. The lancing device preferably includes a lancet and a housing comprising an endcap, the lancet being movable between a first position within the housing and a second position wherein at least a sharp tip portion of the lancet extends through an opening in the endcap. The endcap preferably includes an outwardly-curved contact face for rocking contact with tissue surrounding the sampling site, and at least one slotted opening through the outwardly-curved contact face.

In still another aspect, the invention is a method of collecting a sample of blood or other body fluid, the method preferably including the steps of lancing the skin of a subject at a lancing site with a lancing device having a contact face including one or more surface contour features surrounding an opening; and rocking the lancing device with the contact face in contact with the skin surrounding the lancing site.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a perspective view of an endcap for a lancing device according to one example embodiment of the present invention.

FIGS. 2a and 2b show a perspective view and a side view, respectively, of an endcap for a lancing device according to another example embodiment of the present invention.

FIGS. 3a and 3B show a perspective view and a side view, respectively, of an endcap for a lancing device according to yet another example embodiment of the present invention.

FIG. 4 shows a perspective view of an endcap for a lancing device according to another example embodiment of the present invention.

FIG. 5 shows a perspective view of an endcap for a lancing device according to still another example embodiment of the present invention.

FIG. 6 shows a perspective view of an endcap for a lancing device according to another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

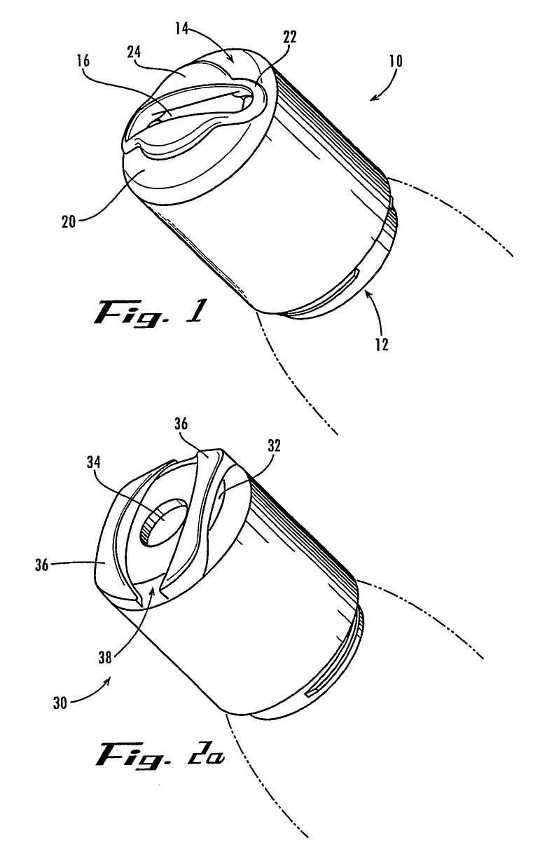

With reference now to the drawing figures, FIG. 1 shows one example embodiment of an endcap 10 for a lancing device according to the present invention. The endcap 10 preferably comprises a first end 12 for releasable or permanent attachment to the remainder of a housing of a lancing device. Alternatively, the endcap forms an integral part of the housing of the lancing device. The endcap 10 can be adapted for use with any of a variety of standard, commercially-available or later-developed lancing devices, by appropriate configuration of coupling or attachment features of the first end 12. The second end 14 of the endcap 10 preferably defines the opening 16 through which the sharp lancet tip projects in its extended position to penetrate the skin at the intended lancing site. In the depicted embodiment, the opening 16 is a slot extending substantially across the second end 14 of the endcap 10. In alternate embodiments, the opening has a circular, triangular, rectangular, polygonal or other shape. The second end 14 of the endcap 10 preferably comprises an outwardly curved or convex contact face 20, with one or more raised surface features formed thereon. For example, in the depicted embodiment, the surface features comprise a raised rim 22 surrounding the slotted opening 16, and a raised side projection 24 extending laterally outward from the contact face 20 along the central portion of both sides of the slot 16. The outer surfaces of the rim 22 and the projection 24 are preferably outwardly radiused to generally match the outwardly curved or convex contour of the contact face 20.

In a preferred method of operation according to the present invention, after firing the lancing device, the user leaves the contact face of the endcap in contact with the skin around the lancing site, and rocks the lancing device, preferably in the plane of the slotted opening and raised rim. The outwardly curved or convex curvature of the contact face facilitates and guides this rocking motion. The device is preferably initially placed with its contact face in contact with the skin at the lancing site and held generally perpendicular to the skin surface during lancing, and then is rocked back and forth in contact with the skin in at least one plane of motion, wherein that plane of motion intersects a lengthwise axis of the overall lancing device and the center of the endcap's lancet opening. The endcap's lancet opening and/or the raised surface feature(s) on the endcap's contact face are preferably generally symmetric along either side of this plane of motion, and the endcap's contact face is curved along this plane of motion, whereby rocking of the lancing device in the plane of motion applies pressure symmetrically about the lancing site. For example, when the endcap embodiment of FIG. 1 is rocked in a plane of motion containing the lengthwise axis of the slotted opening 16, the raised rim 22 and the central projection 24 will apply alternating pressure around the puncture site, directing subcutaneous fluid toward the puncture site and helping prevent premature closure of the puncture site, thereby enhancing collection of increased volume of blood or other body fluid to be sampled.

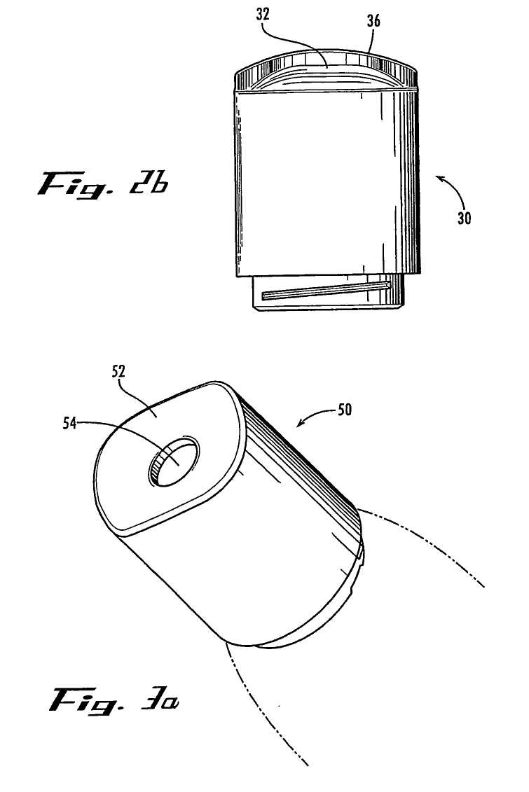

FIGS. 2a and 2b show an endcap 30 for a lancing device according to another embodiment of the present invention. The contact face 32 of this endcap defines a generally circular central opening 34 for allowing passage of the sharp lancet tip. Contact ribs 36 are preferably provided, extending across the contact face 32 along either side of the opening 34. The distal faces of the contact ribs 36 are preferably convexly curved to facilitate and guide rocking of the lancing device in contact with the skin in a similar fashion to that described above. The inner edges of the contact ribs 36 are preferably curved or angled to define a central channel 38 that is wider at its center, around the opening 34, than at its outer edges along the periphery of the endcap. The contours of the contact face 32 and the contact ribs 36 preferably direct subcutaneous fluid toward the puncture site and help prevent premature closure of the puncture site, as the device is rocked back and forth, preferably in a plane of motion extending through the center of the opening and central channel, thereby enhancing collection of increased volume of blood or other body fluid to be sampled.

FIGS. 3a and 3b show an endcap 50 for a lancing device according to yet another embodiment of the present invention. The contact face 52 of this endcap defines a generally circular central opening 54 for passing the sharp lancet tip. The contact face 52 preferably comprises a plurality of alternating raised lobes 56 and depressions 58, forming a continuous, undulating or wave-like surface surrounding the opening 54. After firing, the user toggles, rocks and/or twists the lancing device with the contact face 52 in contact with the skin around the lancing site. The undulating contour of the contact face generates alternating tension and compression of the skin surrounding the lancing site, directing subcutaneous fluid toward the lancing site and helping prevent premature closure of the lancing site, thereby enhancing sample collection.

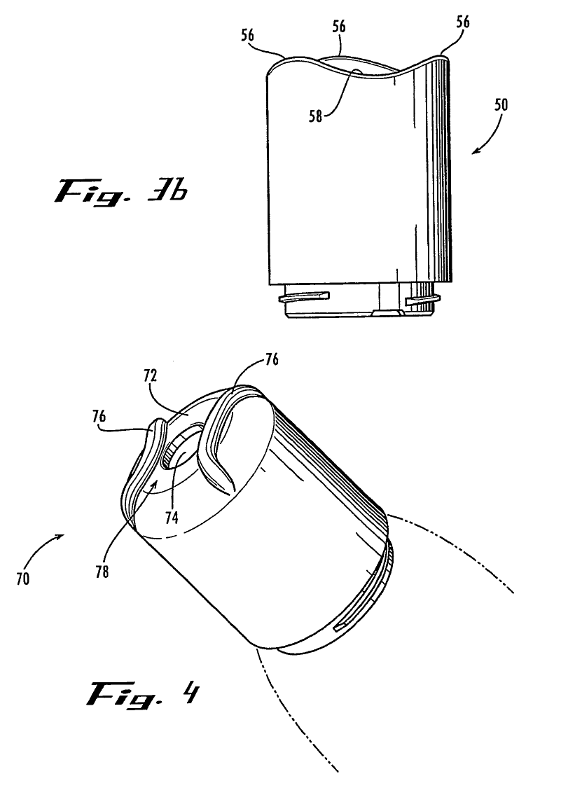

FIG. 4 shows an endcap 70 for a lancing device according to another embodiment of the present invention. The contact face 72 of this endcap defines a generally circular central opening 74 for passing the sharp lancet tip. The contact face 72 preferably comprises a pair of contact ribs 76, extending across the contact face 72 along either side of the opening 74. The distal faces of the contact ribs 76 are preferably convexly curved to facilitate and guide rocking of the lancing device in contact with the skin in a similar fashion to that described above. The inner edges of the contact ribs 76 are preferably curved or angled to define a central channel 78 that is narrower at its center, around the opening 74, than at its outer edges along the periphery of the endcap. The contours of the contact face 72 and the contact ribs 76 preferably direct subcutaneous fluid toward the puncture site and help prevent premature closure of the puncture site, as the device is rocked back and forth, preferably in the plane of the opening and central channel, thereby enhancing collection of increased volume of blood or other body fluid to be sampled.

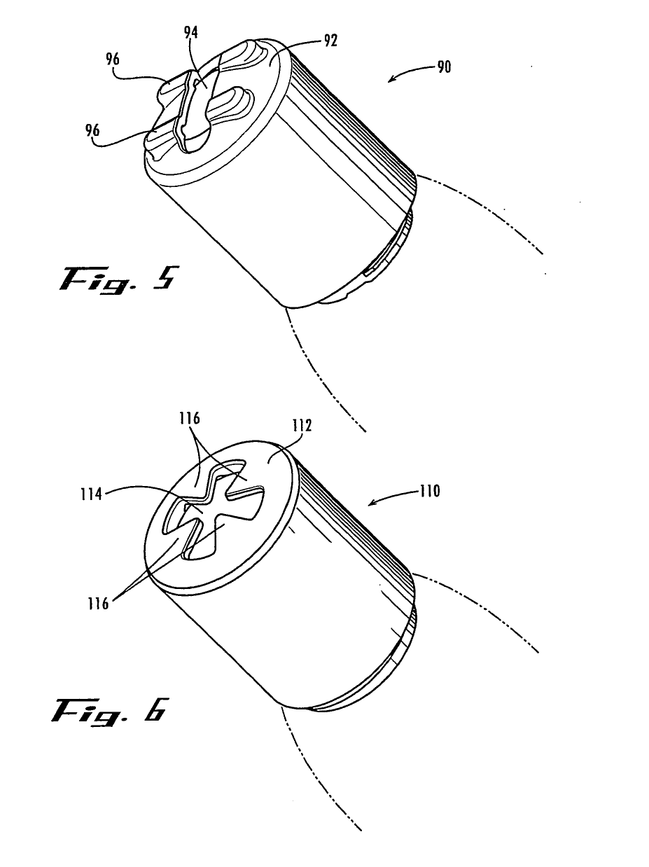

FIG. 5 shows an endcap 90 for a lancing device according to still another embodiment of the present invention. This endcap includes a convex contact face 92 and a slotted opening 94 extending across at least a portion of the width of the contact face. One or more raised ribs or projections 96 are preferably provided, extending across at least a portion of the contact face 92, generally transversely or obliquely oriented relative to the lengthwise dimension of the slot 94. In the depicted embodiment, two raised ribs extend transversely across the slot 94, each rib having a first segment on one side of the slot and a second segment on the other side of the slot. As the lancing device is rocked by the user, preferably in the plane of the slotted opening, the convex contact face 92 and the ribs 96 direct subcutaneous fluid toward the slot 94 and help prevent premature closure of the puncture site, thereby enhancing sample collection.

FIG. 6 shows an endcap 110 for a lancing device according to another embodiment of the present invention. This endcap includes a convex contact face 112 and a cross-shaped opening 114 formed in the contact face. In further preferred forms, the opening 114 is formed by two or more slots intersecting at or near the center of their lengthwise dimensions, each slot being narrower at its central region than at its extremities. In this manner, as the lancing device is toggled or rocked after lancing, preferably in the plane of one or more of the intersecting slots forming the opening 114, subcutaneous fluid is directed and squeezed toward the central intersection of the slots of the opening. In addition, the inwardly-extending lobes 116 between the intersecting slots of the cross-shaped opening 114 can serve as a mechanical stop for the lancet, to limit the path of travel of the lancet at its extended position.

While the invention has been described with reference to preferred and example embodiments, it will be understood by those skilled in the art that a variety of modifications, additions and deletions are within the scope of the invention, as defined by the following claims.

What is claimed is:

1. An endcap for a lancing device, said endcap comprising:
an opening for passage of at least a sharp tip portion of a lancet;
a contact face surrounding said opening; and
at least one surface feature on the contact face adjacent said opening for applying pressure to tissue surrounding a lancing site by rocking of the contact face in contact with the tissue surrounding the lancing site.

2. The endcap of claim 1, wherein a plane of motion is defined through a central lengthwise axis of the endcap and extending through the center of said opening, and wherein the contact face curves outwardly toward said opening along the intersection of the contact face with the plane of motion.

3. The endcap of claim 2, wherein the at least one surface feature is symmetric about said plane of motion.

4. The endcap of claim 1, wherein the at least one surface feature comprises at least one raised rib projecting outwardly from the contact face.

5. The endcap of claim 1, wherein the opening comprises a slotted opening.

6. The endcap of claim 5, wherein said slotted opening is narrower at its central region than at its extremities.

7. The endcap of claim 5, wherein said slotted opening comprises at least two slots intersecting at or near the center of their lengthwise dimensions.

8. The endcap of claim 5, wherein the at least one surface feature comprises a raised rim around at least a portion of said slotted opening.

9. The endcap of claim 8, further comprising a projection extending outwardly from the contact face along the central portion of both sides of the slotted opening.

10. The endcap of claim 5, wherein the at least one surface feature comprises at least one raised rib extending across at least a portion of the contact face, generally transversely or obliquely oriented relative to the lengthwise dimension of the slotted opening.

11. The endcap of claim 1, wherein the at least one surface feature comprises at least one raised rib adjacent the opening.

12. The endcap of claim 11, wherein the at least one raised rib comprises a pair of ribs spaced apart on opposite sides of the opening to define a central channel that is wider at its center than at its extremities.

13. The endcap of claim 11, wherein the at least one raised rib comprises a pair of ribs spaced apart on opposite sides of the opening to define a central channel that is narrower at its center than at its extremities.

14. The endcap of claim 1, wherein the at least one surface feature comprises a plurality of alternating raised lobes and depressions, forming a continuous, undulating contact surface surrounding the opening.

15. An endcap for a lancing device, said endcap comprising an outwardly-curved contact face for rocking contact with tissue surrounding a lancing site, said endcap further comprising at least one slotted opening through the outwardly-curved contact face, the at least one slotted opening being narrower at a central portion than at extremities thereof.

16. The endcap of claim 15, comprising two slotted openings through the outwardly-curved contact face, each slotted opening being narrower at a central portion than at its extremities, wherein the two slotted openings intersect one another at their central portions.

17. A lancing device for collecting a sample of body fluid from a sampling site on the skin of a subject, said lancing device comprising a lancet and a housing comprising an endcap, the lancet being movable between a first position within the housing and a second position wherein at least a sharp tip portion of the lancet extends through an opening in the endcap, wherein the endcap comprises a contact face surrounding the opening and at least one surface feature on the contact face adjacent said opening for applying pressure to tissue surrounding the sampling site by rocking of the contact face in contact therewith.

18. The lancing device of claim 17, wherein a plane of motion is defined through a central lengthwise axis of the lancing device and extending through the center of said opening, and wherein the contact face curves outwardly toward said opening along the intersection of the contact face with the plane of motion.

19. The lancing device of claim 18, wherein the at least one surface feature is symmetric about said plane of motion.

20. The lancing device of claim 17, wherein the at least one surface feature comprises at least one raised rib projecting outwardly from the contact face.

21. The lancing device of claim 17, wherein said opening comprises a slotted opening that is narrower at its central region than at its extremities.

22. The lancing device of claim 17, wherein said slotted opening comprises at least two slots intersecting at or near the center of their lengthwise dimensions.

23. The lancing device of claim 17, wherein the at least one surface feature comprises a plurality of alternating raised lobes and depressions, forming a continuous, undulating contact surface surrounding the opening.

24. A lancing device for collecting a sample of body fluid from a sampling site on the skin of a subject, said lancing device comprising a lancet and a housing comprising an endcap, the lancet being movable between a first position within the housing and a second position wherein at least a sharp tip portion of the lancet extends through an opening in the endcap, said endcap comprising an outwardly-curved contact face for rocking contact with tissue surrounding the sampling site, and at least one slotted opening through the outwardly-curved contact face.

25. The lancing device of claim 24, wherein the at least one slotted opening is narrower at a central portion thereof than at its extremities.

26. The lancing device of claim 24, comprising two slotted openings through the outwardly-curved contact face, the two slotted openings intersecting one another at their central portions.

27. A method of collecting a sample of blood or other body fluid, comprising:
lancing the skin of a subject at a lancing site with a lancing device having a contact face including one or more surface contour features surrounding an opening; and
rocking the lancing device with the contact face in contact with the skin surrounding the lancing site.
